(19) United States
(12) Patent Application Publication
(10) Pub. No.: US 2004/0037857 A1
(43) Pub. Date: Feb. 26, 2004

(54) Anti-pollution composition based on anthocyanic pigments

(76) Inventors: Philippe Catroux, Nogent-sur-Marne (FR); Jose Cotovio, Danmartin-en-Goele (FR); Francis Pruche, Senlis (FR)

Correspondence Address: D Douglas Price, Steptoe & Johnson, 1330 Connecticut Avenue NW, Washington, DC, 20036, US

(21) Appl. No.: 10/276,544

(22) PCT Filed: May 7, 2001
(86) PCT No.: PCT/FR01/01382

(30) Foreign Application Priority Data
May 18, 2000 (FR) 00 06382

(51) Int. Cl. A61K 7/00; A61K 31/7048; A61K 31/353

(52) U.S. Cl. 424/401; 514/27; 514/456

(57) ABSTRACT

The invention concerns the use for topical application of at least an anthocyanic pigment as cosmetic anti-pollution agent, and cosmetic compositions based on anthocyanic pigments and/or plant extract containing an anthocyanic pigment.

The present invention relates to anthocyan-based antipollution compositions, to the use in topical application of anthocyans or extracts containing anthocyans as antipollution cosmetic agents, and also to a cosmetic treatment process using said compositions.

Urban environments are regularly subjected to peaks of pollution. The atmospheric pollutants that are widely represented by the primary and secondary products of combustion represent a major source of environmental oxidative stress. Urban pollution is composed of various types of chemical products, xenobiotics and particles. Three major categories of pollutants can exert deleterious effects on the skin and the hair: gases, heavy metals and particles that are combustion residues, onto which are absorbed a large number of organic compounds.

It is the outermost tissues that are initially and directly exposed to environmental toxic agents. The skin is directly and frequently exposed to the prooxidizing environment. The environmental sources of oxidizing agents include oxygen, solar UV radiation and also, in polluted air, ozone, nitrogen oxides and sulfur oxides. The atmospheric pollutants represented by the primary and secondary products of domestic and industrial combustion such as monocyclic and polycyclic aromatic hydrocarbons are also a major source of oxidative stress. The skin is particularly sensitive to the action of oxidative stress and the outermost layer serves as a barrier against oxidative damage. In most circumstances, the oxidizing agent is likely to be neutralized after reaction with keratin materials, but the reaction products formed may be responsible for attacks on cells and tissues.

The stratum corneum, which is the skin's barrier, is the site of contact between the air and skin tissue. The lipid/protein two-phase structure is a crucial factor of this skin barrier function. These elements can react with oxidizing agents and be impaired, which will promote the phenomena of desquamation. Ozone-induced lipid peroxidation can impair the skin in two ways:

1/ The oxidation and degradation of the lipids of the stratum corneum can impair the barrier function of the stratum corneum. Disruption of the outer lipids and of the protein architecture appear to be triggering factors in many dermatoses (psoriasis, atopic dermatitis and irritant dermatitis).

2/ The increased formation of lipid-oxidation products in the upper layers of the skin can trigger attacks in the adjacent layers of skin. The reaction of ozone (O₃) with unsaturated lipids involves addition reactions onto the double bonds. This process leads in a second stage to cleavage of the lipid chains and to the formation of aldehyde hydroperoxides and of hydrogen peroxide.

This involves a specific mechanism that is different from the lipoperoxidation mechanism conventionally described, which is mediated by a radical. The ozone-induced products of secondary or tertiary lipid oxidation, which are less reactive than ozone but have a longer lifetime, can propagate the effect of ozone. On account of their relative stability, the products of lipid oxidation and peroxidation, i.e. cholesterol oxides and aldehydes, have the potential to impair cells at remote sites not directly exposed to O₃.

A significant level of oxidative attack on the surface layers of the stratum can initiate localized subjacent inflammatory processes, leading to the recruitment of phagocytes, which, by generating oxidizing agents, will amplify the initial oxidative processes.

In urban pollution, concomitant exposure to O₃ and to UV can cause synergistic oxidative stress.

Similarly, it may be thought that there is synergistic action between ozone and combustion-derived organic compounds.

Among the pollutants that can exert deleterious effects on keratin materials, toxic gases, such as ozone, carbon monoxide, nitrogen oxides or sulfur oxides, are the major constituents of the pollutants.

It has been found that these toxic gases promote the desquamation of keratin materials and “fatigue” said keratin materials, i.e. make them dirty and dull. Similarly, cellular asphyxia of said keratin materials has been found.

Thus, there is a need for compositions for preventing the harmful effects caused by these pollutants, thus making it possible to protect keratin materials.

Moreover, patent U.S. Pat. No. 4,503,037 describes therapeutic compositions containing, inter alia, anthocyans, used for therapeutic purposes to treat skin burns.

It has now been found, entirely surprisingly, that the use of anthocyans allows keratin materials to be protected against the effects of pollutants.

Anthocyans are found in plants in the form of heterosides, anthocyanosides, acid hydrolysis of which releases the genins (or aglycone, known as anthocyanidole). These pigments are a more or less dark red in acidic solution and turn blue or violet-blue in the presence of alkali.

In the context-of the present invention, the anthocyans used may be pure, for example obtained via biotechnology.

They may also be used in isolated form from various extracts. Examples that may especially be mentioned include extracts of bilberry fruit, the fruit of European elder syrup or Mediterranean herb elder syrup, blackberry fruit, blackcurrant, sweet cherry fruit, blackthorn fruit, grapes, creeper grapes, privet, privet fruit, and also rose petals, poppy, corn poppy or alternatively sorghum seeds or rohan soymida, sappan wood, Brazilian rosewood, pernambuco wood, Jamaica quassia wood or cocobolo, red sandalwood, padauk, redwood, muninga and camwood.

Thus, the main subject of the invention is the use in topical application of at least one anthocyan as defined above, as an antipollution cosmetic agent.

The expression “antipollution cosmetic agent” means an agent that protects the skin and keratin materials so as to prevent, attenuate and/or suppress the deleterious effects of toxic gases such as ozone.

These anthocyans may especially correspond to those of formula (I):

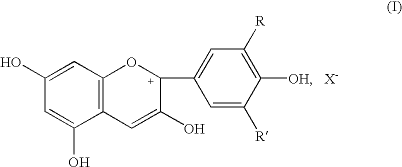

in which:

R and R′ represent, independently of each other, a hydrogen atom, hydroxyl radical or a C1-C6 alkoxy or oside group; X⁻ representing a monovalent counteranion.

Preferably, R and R′ represent a hydrogen atom, a hydroxyl, a methoxy or a glucoside and X⁻ represents a halogen atom.

Preferred anthocyans that may especially be mentioned include malvidol, paenidol, petunidol, delphinidol and oenoside. The latter anthocyan is most particularly preferred.

The plant extract containing at least one anthocyan as defined above may also be used in topical application as an antipollution topical agent.

The anthocyan or the extract containing at least one anthocyan as defined above is used especially in order to protect keratin materials against the effects of toxic gases.

In the context of the present invention, the expression “keratin material” especially means the skin, the scalp, the hair, the eyelashes, the eyebrows, the nails and mucous membranes.

The anthocyans and/or the extract containing anthocyans may also be used to improve cell respiration and/or to reduce desquamation and/or to prevent keratin materials from becoming dull or dirty.

A subject of the invention is also a nonpolluting cosmetic composition for topical application which may be in the presentation forms normally used for topical application, especially in the form of an oil-in-water, water-in-oil or multiple emulsion, an aqueous or oily gel, a dispersion of oil in an aqueous phase with the aid of spherules or lipid vesicles of ionic and/or nonionic type.

The compositions according to the invention contain, in a cosmetically acceptable medium, at least one anthocyan and or an extract of anthocyan as defined above.

This composition may especially contain from 0.005% to 10% and preferably from 0.1% to 5% by weight of active material of anthocyan active material relative to the total weight of the composition.

This composition may also contain at least one other antipollution compound. Said compound may be chosen especially from compounds containing a thioether, sulfoxide or sulfone function, ergothioneine and/or its derivatives, metal-chelating agents such as, for example, N,N′-dibenzylethylenediamine-N,N′-diacetic acid derivatives, ellagic acid or a cell extract of a plant of the Pontederiacea family.

The cosmetically acceptable medium for the compositions according to the invention consists more particularly of water and/or optionally of a cosmetically acceptable organic solvent.

The organic solvents may represent from 5% to 98% of the total weight of the composition. They may be chosen from the group consisting of hydrophilic organic solvents, lipophilic organic solvents and amphiphilic solvents, or mixtures thereof. Among the hydrophilic organic solvents that may be mentioned, for example, are monofunctional or polyfunctional alcohols such as linear or branched lower monoalcohols containing from 1 to 8 carbon atoms, for instance ethanol, propanol, butanol, isopropanol and isobutanol; optionally oxyethylenated polyethylene glycols containing from 6 to 80 ethylene oxides, polyols such as propylene glycol, isoprene glycol, butylene glycol, glycerol, sorbitol and its derivatives, monoalkyl or dialkyl isosorbide, the alkyl groups of which contain from 1 to 5 carbon atoms, for instance dimethyl isosorbide, glycol ethers, for instance diethylene glycol monomethyl or monoethyl ether, and propylene glycol ethers, for instance dipropylene glycol methyl ether.

Amphiphilic organic solvents that may be mentioned include polyols such as derivatives of propylene glycol (PPG), such as esters of polypropylene glycol and especially esters of polypropylene glycol and of fatty acids, derivatives of PPG and of fatty alcohols, for instance PPG-23 oleyl ether, and PPG-36 oleate.

Lipophilic organic solvents that may be mentioned, for example, include fatty esters such as diisopropyl adipate, dioctyl adipate and alkyl benzoates.

In order for the cosmetic or dermatological compositions used in the invention to be more pleasant to use (softer to apply, more nourishing and more emollient), it is possible to add a fatty phase to the medium of these compositions.

The fatty phase preferably represents from 0% to 50% relative to the total weight of the composition.

This fatty phase may comprise one or more oils preferably chosen from the group consisting of:

volatile or nonvolatile, linear, branched or cyclic, organomodified or non-organomodified, water-soluble or liposoluble silicones,

mineral oils such as liquid paraffin and liquid petroleum jelly,

oils of animal origin such as perhydrosqualene,

oils of plant origin such as sweet almond oil, avocado oil, castor oil, olive oil, jojoba oil, sesame oil, groundnut oil, macadamia oil, grape-seed oil, rapeseed oil or coconut oil,

synthetic oils such as purcellin oil and isoparaffins,

fluoro oils and perfluoro oils,

fatty acid esters such as purcellin oil

Said fatty phase may also comprise as fatty substances one or more fatty alcohols, fatty acids or waxes (paraffin wax, polyethylene wax, carnauba wax or beeswax).

In a known manner, the compositions used in the invention may also contain adjuvants that are common in cosmetics, such as standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, dyestuffs, bactericides and antidandruff agents.

The amounts of these various adjuvants are those conventionally used in the fields under consideration.

Needless to say, a person skilled in the art will take care to select the optional compound(s) to be added to the composition according to the invention, such that the advantageous properties intrinsically associated with the composition in accordance with the invention are not, or are not substantially, adversely affected by the envisioned addition.

The compositions according to the invention may be more or less fluid and may have the appearance of a white or colored cream, an ointment, a milk, a lotion, a serum, a paste or a mousse.

They may optionally be applied to the skin in aerosol form.

They may also be applied in solid form, and for example in the form of a stick.

They may be used as care products and/or as makeup products.

The compositions according to the invention may have a pH of between 3 and 8 and preferably between 5 and 7.

A subject of the invention is also the use of at least one anthocyan or of an extract as defined above, in or for the preparation of an antipolluting composition for topical application.

A subject of the invention is also the use of at least one anthocyan or of an extract as defined above as a colorant in or for the preparation of an antipolluting makeup composition for topical application.

Another subject of the invention consists of a cosmetic treatment process for protecting the body against the effects of pollution, which consists in applying to the keratin materials a cosmetically effective amount of at least one anthocyan or an extract containing an anthocyan as defined above.

Another cosmetic treatment process according to the invention, for protecting the body against the effects of Pollution, consists in applying to the keratin materials a cosmetic composition according to the invention, as defined above.

The examples that follow are intended to illustrate the invention without, however, being limiting in nature.

### EXPERIMENTS

1) Pretreatment of keratinocytes with an extract of powdered grape skin sprayed onto maltodextrin, having an anthocyan titer (1250 CU/G), sold by the company Hansen under the name “Extrait de raisin ANT 1250 P”. [Grape extract ANT 1250 P]

A solution containing 125 μg/ml of the extract in the culture medium is prepared extemporaneously.

Placing in contact with the cells is performed (500 μl/well for 24 hours).

2) Incorporation of a marker: 2,7-dichlorofluorescin diacetate.

Remove the protective solution, rinse with phosphate-buffered saline PBS,

Place in contact for 30 minutes with a solution of 2,7-dichlorofluorescin diacetate (320 μM in PBS), 500 μl/well.

Rinse with PBS.

3) Exposure to Ozone

Prepare a fresh protective solution in the culture medium at the maximum non-cytotoxic concentration, i.e. 250 μg/ml of extract,

Deposit 100 μl per well of this solution,

Expose the cells to ozone in an incubator (37° C., humid atmosphere): ozone concentration: 10 ppm.

Measurement of the effects induced by ozone

Measure the appearance of 2,7-dichlorofluorescine (DCF) (fluorescence excitation: 485, emission: 530) after different exposure times: 0, 5, 10, 20 minutes.

RESULTS

Number of independent experiments: 4

Number of repetitions per experiment: 6

Toxicity of ozone towards human keratinocytes, in the absence or presence of extract of grape skin at a concentration of 250 μg/ml, as a function of the exposure time (n=4).

Fluorescence observed in the presence of extract of grape
skin, expressed as % relative to the unprotected controls
for each time

| 5 min. of contact | 10 min. of contact | 20 min. of contact |
|---|---|---|
| % fluorescence | % fluorescence | % fluorescence |
| observed ± | observed ± | observed ± |
| SEM | SEM | SEM |
| 45.4 ± 6.1 | 60.3 ± 4.8 | 76.5 ± 2.6 |

The appearance of ozone-generated peroxides in the cell is reflected by an increase in the fluorescence. For each time, the fluorescence values of the unprotected controls are set at 100%. The results, in the presence of extract of grape skin, are then expressed relative to this control value. A reduction in this value indicates a protective effect of the extract.

### CONCLUSIONS

In the presence of a high concentration of ozone, the anthocyan-based extract significantly reduces the effects induced by ozone. This protection is at a maximum from 5 minutes of exposure onward (54.9% drop in induced stress). It is still significant after 20 minutes of exposure (23.5% drop in induced stress).

### FORMULATION EXAMPLES

### Example 1

According to the usual preparation techniques, the constituents below are mixed together to prepare an emulsion.

| | | |
|---|---|---|
| oenoside | 0.1 | g |
| polyethylene glycol oxyethylenated with | 3 | g |
| 50 mol of ethylene oxide | | |
| monodiglyceryl stearate | 3 | g |
| liquid petroleum jelly | 24 | g |
| cetyl alcohol | 5 | g |
| water | qs 100 | g |

### Example 2

In the same manner, an emulsion is prepared according to a standard technique, using the following compounds:

| | | |
|---|---|---|
| malvidol | 0.02 | g |
| octyl palmitate | 10 | g |
| glyceryl isostearate | 4 | g |
| liquid petroleum jelly | 24 | g |
| vitamin E | 1 | g |
| glycerol | 3 | g |
| water | qs 100 | g |

### Example 3

Starting with the constituents below, the following composition is formulated:

| | | |
|---|---|---|
| delphinidol | 1 | g |
| jojoba oil | 13 | g |
| methyl isopropyl para-benzoxybenzoate | 0.05 | g |
| potassium sorbate | 0.3 | g |
| cyclopentadimethylsiloxane | 10 | g |
| ellagic acid | 0.1 | g |
| stearyl alcohol | 1 | g |
| stearic acid | 4 | g |
| polyethylene glycol stearate | 3 | g |
| vitamin E | 1 | g |
| glycerol | 3 | g |
| water | qs 100 | g |

### Example 4

Starting with the constituents below, the following composition is formulated:

| | | |
|---|---|---|
| oenoside | 0.1 | g |
| jojoba oil | 13 | g |
| methyl isopropyl para-benzoxybenzoate | 0.05 | g |
| potassium sorbate | 0.3 | g |
| cyclopentadimethylsiloxane | 10 | g |
| N,N′-bis (3-hydroxybenzyl)ethylenediamine- | 0.1 | g |
| N,N′-diacetic acid | | |
| stearyl alcohol | 1 | g |
| stearic acid | 4 | g |
| polyethylene glycol stearate | 3 | g |
| vitamin E | 1 | g |
| glycerol | 3 | g |
| water | qs 100 | g |

1. Use in topical application of at least one anthocyan as an antipollution cosmetic agent.

2. The use as claimed in claim 1, characterized in that the anthocyan corresponds to formula (I): 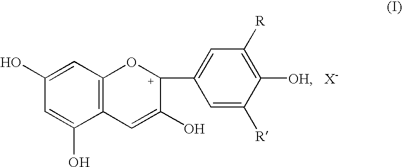
in which:
R and R′ represent, independently of each other, a hydrogen atom, hydroxyl radical or a C1-C6 alkoxy or oside group;
X⁻ representing a monovalent counteranion.

3. The use as claimed in claim 2, in which R and R′ represent, independently of each other, a hydrogen atom, hydroxyl radical or a methoxy or glucoside group and X⁻ represents a halogen atom.

4. The use as claimed in any one of claims 1 to 3, characterized in that the anthocyan is chosen from malvidol, paenidol, petunidol, delphinidol and oenoside.

5. The use as claimed in any one of claims 1 to 4, characterized in that the anthocyan is an oenoside.

6. The use in topical application of at least one plant extract containing at least one anthocyan as defined in any one of claims 1 to 5, as an antipollution cosmetic agent.

7. The use in topical application of at least one anthocyan as defined in any one of claims 1 to 5 or of a plant extract containing at least one anthocyan as defined in claim 6, as a cosmetic agent for protecting keratin materials against the effects of toxic gases.

8. The use in topical application of at least one anthocyan as defined in any one of claims 1 to 5 or of a plant extract containing at least one anthocyan as defined in claim 6, as a cosmetic agent for improving cell respiration and/or for reducing desquamation and/or for preventing keratin materials from becoming dull or dirty.

9. An antipolluting cosmetic composition for topical application, characterized in that it is in the form of an oil-in-water, water-in-oil or multiple emulsion, an aqueous or oily gel, or a dispersion of oil in an aqueous phase with the aid of spherules or lipid vesicles, and in that it contains, in a cosmetically acceptable medium, at least one anthocyan as defined in any one of claims 1 to 5 or a plant extract containing at least one anthocyan as defined in claim 6.

10. The composition as claimed in claim 9, characterized in that said antipolluting cosmetic composition contains from 0.005% to 10% and preferably from 0.1% to 5% by weight of anthocyan active material relative to the total weight of the composition.

11. The composition as claimed in claim 9 or 10, characterized in that said composition also contains at least one other antipollution compound.

12. The composition as claimed in claim 11, characterized in that said antipollution compound is chosen from compounds containing a thioether, sulfoxide or sulfone function, ergothioneine and/or its derivatives, metal-chelating agents such as, for example, N,N′-dibenzylethylenediamine-N,N′-diacetic acid derivatives, ellagic acid or a cell extract of a plant of the Pontederiacea family.

13. The composition as claimed in any one of claims 9 to 12, characterized in that the cosmetically acceptable medium consists of water and/or of at least one organic solvent chosen from the group consisting of hydrophilic organic solvents, lipophilic organic solvents and amphiphilic solvents, or mixtures thereof.

14. The composition as claimed in claim 13, characterized in that the organic solvents are chosen from the group consisting of monofunctional or polyfunctional alcohols, optionally oxyethylenated polyethylene glycols, polypropylene glycol esters, sorbitol and its derivatives, dialkyl isosorbides, glycol ethers and polypropylene glycol ethers, and fatty esters.

15. The composition as claimed in claim 13 or 14, characterized in that the organic solvent(s) represent(s) from 5% to 98% relative to the total weight of the composition.

16. The composition as claimed in any one of claims 9 to 15, characterized in that it also comprises at least one fatty phase.

17. The composition as claimed in claim 16, characterized in that the fatty phase represents from 0% to 50% relative to the total weight of the composition.

18. The composition as claimed in any one of claims 9 to 17, characterized in that it also contains at least one additive chosen from the group consisting of standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, dyestuffs, bactericides and anti-dandruff agents.

19. The composition as claimed in any one of claims 9 to 18, characterized in that the composition has the appearance of a white or colored cream, an ointment, a milk, a lotion, a serum, a paste, a mousse or a solid.

20. The composition as claimed in any one of claims 9 to 19, characterized in that it has a pH of between 3 and 8 and preferably between 5 and 7.

21. The use of an anthocyan as defined in any one of claims 1 to 5, or of a plant extract containing at least one anthocyan as defined in claim 6, in or for the preparation of an antipolluting composition for topical application.

22. The use of an anthocyan as defined in any one of claims 1 to 5, or of a plant extract containing at least one anthocyan as defined in claim 6, as a colorant in or for the preparation of an antipolluting makeup composition for topical application.

23. A cosmetic treatment process for protecting the body against the effects of pollution, characterized in that it consists in applying to keratin materials a cosmetically effective amount of at least one anthocyan as defined in any one of claims 1 to 5, or of a plant extract containing at least one anthocyan as defined in claim 6.

24. A cosmetic treatment process for protecting the body against the effects of pollution, characterized in that it consists in applying to keratin materials a composition as defined in any one of claims 9 to 20.
